(19) United States
(12) Patent Application Publication
(10) Pub. No.: US 2007/0207115 A1
(43) Pub. Date: Sep. 6, 2007

(54) Tea Tree Oil and Benzoyl Peroxide Acne Treatment

(76) Inventor: Nanette Liegeois, Lutherville, MD (US)

Correspondence Address: GREENBERG & LIEBERMAN, LLC, 2141 WISCONSIN AVE, N.W., SUITE C-2, WASHINGTON, DC, 20007, US

(21) Appl. No.: 11/307,969

(22) Filed: Mar. 1, 2006

(51) Int. Cl. A61K 8/97; A61K 31/192; A61K 36/61

(52) U.S. Cl. 424/74; 424/769; 514/568

(57) ABSTRACT

A system is disclosed consisting of tea tree oil used alone or in combination with conventional over-the-counter strength anti-acne agents. A system is created whereby Tea tree oil is used in combination with other anti-acne therapies, particularly benzoyl peroxide. Subsequent steps utilize conventional anti-acne treatments.

### FIELD OF THE INVENTION

The present invention is an acne treatment, and more particularly, is an acne treatment with tea tree oil in combination with benzoyl peroxide.

### BACKGROUND OF THE INVENTION

Acne is a condition of the skin that can occur at any age. Although acne can sometimes be a cosmetic concern, acne can also be disfiguring and scarring. The changes in the skin that are present even after acne resolves are often permanent or difficult to correct. Evidence that acne poses a major health problem in the United States is that it is one of the top reasons for people to visit their dermatologist. There are three types of acne: mild, moderate and severe. Mild acne is characterized by open and closed comedones (called white and blackheads in layman's terms). Moderate acne is characterized by more inflammatory lesions, with redness, pus, and the potential for significant scarring if left untreated (“pock marks”). Severe acne typically includes nodules, cysts and painful lesions that lead to disfiguring scars. There are several effective modalities to treat mild and moderate acne that include topical as well as oral preparations.

Acne is a sebaceous gland disorder that is the clinical result of a multifactorial process. There are many physiologic reasons for acne to occur: 1) hormones 2) occlusion 3) genetic 4) bacteria. Hormonal changes, such as those that occur during adolescence, are a well-known cause of acne. Other hormonal influences are also known to be important for developing acne, such as the fluctuation in hormones that occurs for women during menstruation; this “time of the month” can be associated with increased acne. Additionally, there are syndromes whereby a serious imbalance of hormones results in clinical acne. Occlusion of pores is a known etiologic factor in the development of acne. Exfoliants are products that take away “dead skin” and decrease acne by unclogging these pores. Finally, genetic predisposition is often at play with acne. Many times, a person has a significant family history of acne and their own personal experience mimics the experience of their other family members. Propionibacterium acnes (P. acnes) is thought to be the culprit bacteria contributing to acne and many effective therapies, such as topical benzoyl peroxide or antibiotics are directed toward eradicating P. acnes. Unfortunately, many of the traditional therapies used to treat these four major causes of acne have unfortunate and/or irritating side effects.

Topical therapy is the first line of therapeutic treatment for acne. Topical benzoyl peroxide is extremely effective in treating acne and is the workhorse for many anti-acne therapies. Topical benzoyl peroxide, however, is limited because high concentrations needed to thoroughly treat the acne are often not achieved because such concentrations are irritating to the skin or leave it dry. When the skin becomes dry, patients tend to apply more moisturizers and this leads to occlusion of the pores, resulting in increased acne. The irritation of topical therapy is the limiting factor in maximizing treatment. Additionally, bacterial resistance can occur in any anti-acne therapy.

When topical treatment fails to improve the acne condition, many physicians recommend oral therapy. Oral antibiotics are commonly used and prescribed by physicians in order to treat acne. Although quite effective, their side effects can be significant and lead to discontinuance. Oral antibiotics have proven to be efficient against acne; however, concern for the development of drug resistant bacteria (Bojar and Holland 2004) and the possibility exists that such use will predispose to breast cancer (Stamp 2004; Velicer, Heckbert et al. 2004). It is well known that chronic use or prolonged use of antibiotics can lead to yeast and fungal infections. Additionally, oral antibiotics can create serious drug interactions and potentially life-threatening situations with commonly prescribed medications, including medications for conditions such as heart disease, heartburn, asthma, and birth control pills. Other side effects of commonly used acne antibiotics include side effects that are not life threatening but are very serious. This includes one of the more commonly used antibiotics, tetracycline, which has the side effect of causing permanent discoloration of the teeth if prescribed to pregnant women or children under 8 years old. Additionally, antibiotics increase the sensitivity of sun and can lead to serious sunburn. Severe kidney damage or failure can occur when antibiotics are used. Although all of these are very serious, the most common side effect for many people who take oral antibiotics is severe nausea and/or diarrhea. Given all of these concerns, there is a need for improved topical therapy; specifically, there is a need to maximize the effectiveness of topical therapy. In order to accomplish anti-inflammatory activity into a regimen that embodies effective anti-acne therapy, there is a need for a new topical therapy system that incorporates anti-inflammatory properties in its formulation. Specifically, there is a need to maximize the effectiveness of standard therapies (that would otherwise be irritating) while decreasing irritation.

Retinoids have very effective anti-acne effects. Topical retinoids, such as adapalene, tretinoin, and tazarotene are effective at treating acne. Their function is to exfoliate and unclog pores. Topical retinoids have been shown to improve photodamage and redness as well. Serious skin irritation is an extremely common side effect, however, often leading to discontinuance of the product (Samuel, Brooke et al. 2005). Oral retinoids, such as isotretinoin, are extremely effective against acne. Unfortunately, they have extremely high rates of teratogenicity (birth defects) so that their prescribing method is very strictly regulated and needs to be saved for only the most severe forms of acne. Accutane is a serious teratogen and causes major fetal abnormalities (Morrison, Elsas et al. 2005). For these severe cases, caution is of value since the list of side effects is very long and includes visual disturbance, headache, bone changes, poor wound healing, pancreas and liver dysfunction, and depression. There have been frequent reports of suicide attempts or ideation in patients taking Accutane (2003). The FDA keeps a database of drugs associated with depression and suicide attempts. Accutane is listed in the top 10 of these drugs (Barak, Wohl et al. 2005). A serious mental condition—manic psychosis—has been correlated with the use of Accutane and those cases have been associated with suicide attempts (Barak, Wohl et al. 2005). Because of all the side effects, the FDA is requiring special safety training for physicians who might prescribe Accutane (Honein, Moore et al. 2004; Brinker, Kornegay et al. 2005). Studies using MRI of the brain have demonstrated that Accutane causes a measurable change in brain function (Bremner, Fani et al. 2005). Given the concern for birth defects and possibility of depression, there is a need to have anti-acne therapy without these products.

Oral contraceptives are occasionally used to treat acne. Although they can be quite effective, particularly in hormonally exacerbated acne, contraceptives can cause blood clots, stroke, and possibly death. Additionally, women over the age of 35 should not be on long-term oral contraceptives. There is a need to have topical therapy for treatment of acne without the total body side effects associated with oral therapy.

Many of the over-the-counter agents used for fighting acne have the side effect of causing irritation. An effective anti-acne treatment would be truly beneficial if it also reduced the irritation from the anti-acne agent. There are many products, such as topical steroids, that can diminish or prevent irritation of the skin, however chronic application of topical steroid actually causes acne and can result in irreversible (and permanent) thinning of the skin. Even short-term topical steroid application to the face is not recommended because of the serious side effects that can occur.

Users of acne treatments are often quick to judge treatment results, even though perceived or real improvement in skin appearance might take 6 weeks to 3 months. There is a real need to allow users to see skin improvement shortly after using an acne treatment; else the users might abandon the acne treatment, as they are looking for the quickest remedy available to eradicate acne. Users don't always have the patience or the habits to use a product consistently over a period of time—even though consistent use of an acne-fighting product over time might yield desirable results. Quite simply, users want results instantaneously, without having to take time out day after day to alter lifestyle for acne treatment. There is a need to provide an acne treatment that calls for high concentrations of acne combating substances to come into contact with skin that has fallen to acne, while at the same time, ensuring that the skin does not become irritated.

In describing the various embodiments of the present invention, the term “conventional anti-acne treatment(s)” is used. For the purpose of the present invention, “conventional anti-acne treatment(s)” and similar terms should be considered as all-inclusive for anti-acne therapy/ies. The management, treatment, and prevention of acne, including both over-the-counter and prescription remedies that contain effective anti-acne ingredients, are described. These concern (but are not limited to) products containing: salicylic acid, benzoyl peroxide, ascorbic acid and derivatives, alpha hydroxyl acids, beta hydroxyl acids, benzoyl peroxides, Vitamin C derivaties, Vitamin A derivatives. These types of products are marketed by many companies and are considered “conventional anti-acne therapies” although they exist in many formulations and are sold by many companies including (but not limited to): alpha-hydroxy acids, benzoyl peroxide, salicylic acid, glycolic acid or retinoids. Conventional acne medications also include therapies that require a prescription; these include topical (and oral) antibiotics as well as topical (and oral) retinoids. Examples of antibiotics are erythromycin, doxycycline, tetracycline, and clindamycin. Examples of prescription strength retinoids include topical tazarotene, adapalene, and tretinoin as well as oral isotretinoin. Anti-acne therapies can be combined with other therapies for conditions such as pseudofolliculitis barbae, anti-aging, anti-inflammatory, and anti-pigmentation. Currently marketed anti-acne products are in some cases aimed at other treatments in addition to combating acne.

Tea tree oil has been described as a natural method of topically fighting acne. Although tea tree oil would appear to hold much promise as a single agent topical treatment for acne, there are many reasons why this is not optimal. First people want quick results. Tea tree oil takes far longer than other treatments to reduce acne. Second, direct application of tea tree oil extract to the skin is not tolerable in high concentrations as the odor of high concentration tea tree extract is very sharp and pungent. Finally, the consistency is not suitable as an anti-acne agent because it leaves a perceptible residue on the skin that feels like a film; this is precisely the texture that acne patients try to relieve rather than induce. Tea tree oil high concentrate extract takes too long to relieve acne conditions and further leaves a residue and offensive odor that induces patients to desire that they cleanse their skin. Alas, this side effect precludes the use of tea tree oil concentrate as an option for being a single agent in treating acne.

While the Martin and Ernst study did show marked improvement in treating acne with tea tree oil in comparison to conventional benzoyl peroxide, it should be noted that the concentrations of tea tree oil employed were conservative. Thus, to use tea tree oil safely, without irritating the very skin that the user seeks to improve, tea tree oil in moderation is the only answer currently. However, it is desirable to be able to treat acne with more effectiveness than that achieved with a moderate level of tea tree oil.

Any person with acne wants to eradicate the acne as soon as possible, and that means applying high concentrations of benzoyl peroxide repeatedly to skin suffering from acne. However, a high concentration of benzoyl peroxide is extremely irritating; moreover and high concentration of tea tree oil can be likened to “applying a layer of turpentine to the skin”. Tea tree oil, while effective against acne, causes displeasing results when applied in high concentrations, as does benzoyl peroxide. Ideally, it is desirable to apply benzoyl peroxide in topical form (as a cleanser, lotion, toner, etc) to skin suffering from acne twice per day at highest concentration tolerable. This is because anti-acne therapy is most effective when it is repeated frequently and at high concentrations. As stated previously, while high concentration of tea tree oil is noxious, high concentration of benzoyl peroxide is prohibitive because of adverse reactions of irritation.

A need has been established for an efficacious method for treating acne with an improved combination of known compounds that allow standard topical therapy to be maximized. While there are well described anti-inflammatory and anti-acne properties of natural therapy alone or in combination with traditional acne therapy, there is heretofore no product that attempts to provide anti-inflammatory factors to allow maximal topical therapy.

### SUMMARY OF THE INVENTION

The present invention is based, at least in part, on the discovery that topical Tea tree oil extract has anti-acne effects as well anti-inflammatory properties. The anti-inflammatory effects are from direct action against the cells in the body that are known to produce redness and irritation in response to environmental assault. While tea tree oil is gaining popularity and can be perceived as conventional in treating certain irritating conditions, it is a unique aspect of the present invention that tea tree oil is used to treat and prevent the irritation from a conventional benzoyl peroxide (BPO) cleansing treatment, such that BPO can be used in higher concentration and with higher frequency, producing less irritation. This application has not yet been described for tea tree oil.

In comparison studies, Tea tree oil extracts have both been shown to be as effective as standard over-the-counter strength topical benzoyl peroxide. Additionally, these agents have the added benefit of being less irritating than standardly used over-the-counter topical benzoyl peroxide. In separate studies, the ingredients in Tea tree oil appear to have antibacterial properties against many types of bacteria. Important for the present invention, it effectively fights the bacteria responsible for acne: P. acnes. The constituents involved in fighting the acne include the terpinen constituents, that include alpha-terpineol, terpinen-4-ol, and alpha-pinene.

The present treatment protocol is efficient, topical, and provides an anti-acne regimen that provides an effective treatment. It additionally provides less irritation compared with standard over-the-counter therapies. Such a system relies on tea tree oil as one of its ingredients and such ingredient allows for this system to be less irritating than traditional treatments. It also includes standard over-the-counter remedies that are currently used to cure or minimize acne and the clinical sequelae (including scars and discoloration).

The amount of Tea tree oil to treat acne is not fixed in the present invention, and will depend on other active ingredients also found in the solutions as well as the patient's acne condition. Different types of acne may be found to respond with variable results. As well, the amount of improvement may depend on the patient's skin type and prior scarring. Since efficacy is diminished below 0.05% and the concentration above 50% is displeasing (it leaves a film on the person's skin), concentrations up to 50% are included in this invention. As examples in this application, a 4% by volume tea tree oil composition is described. The exact percent can vary depending on the carrier, solvent, and other anti-acne ingredients. The usual formulation is an aqueous solution with a carrier described below. In the examples that follow, efficacious compositions illustrating the invention contain a 4% aqueous solution of tea tree with PEG surfactant.

Tea tree oil is an extract from the melaleuca alternifolia tree, which readily grows in Australia, New Zealand, South Wales, Queensland, and the East Indies. It contains several compounds that have anti-inflammatory properties. Known active compounds present in the extract include alpha-terpineol, terpinen-4-ol, and alpha-pinene.

Why it works: It has been shown to have anti-inflammatory activity at the cellular level; has gained popularity in treating inflammatory (red) conditions of the skin, such as insect stings and bites, burns, and skin abrasions; has the additional benefit of having antibacterial activity so it has added benefit in this invention of anti-acne therapy; and finally, it produces very little irritation to the skin.

Tea tree oil has an anti-inflammatory effect when applied topically. The soothing properties of tea tree oil are based on scientific studies that show that tea tree oil compounds have a significant effect in diminishing the inflammatory response. It is a unique design of the present invention that the anti-inflammatory properties of tea tree oil are incorporated into the anti-acne formulation in such a way as to counteract the inflammatory property of conventional anti-acne therapy. Specifically, the leading anti-acne cleanser, benzoyl peroxide, is one of the most effective anti-acne therapies; prolonged high dosing is limited by the irritating side effects. Many systems incorporate the cleanser into their product, as it is an effective cleanser. Because benzoyl peroxide is a mainstay in anti-acne therapy and is the lead player in anti-acne cleansers, the tea tree oil product is specifically combined with benzoyl peroxide to reduce the inflammatory nature of benzoyl peroxide based cleansers. To date, there are no anti-acne formulations that incorporate the anti-inflammatory properties of tea tree oil in such a way in order to counteract the inflammatory properties of conventional anti-acne treatment. Compatibility of benzoyl peroxide and tea tree oil in a combined formulation allows for the anti-inflammatory activities of tea tree oil to work at the same time as the cleansing properties of benzoyl peroxide.

In the present invention, tea tree oil has the added advantage that it has anti-acne properties as well. Additionally, many people prefer to avoid the use of “chemicals” in traditional medical treatments in favor of “natural” therapies and so that there is the advantage of perceived value to including “natural” therapies with conventional treatments.

Tea tree oil, extracted from the Melaleuca alternitolia, has multiple components that are thought to be active in the process of treating acne. These, as well as other herbal treatments, are efficacious for the treatment of acne and some are improved over benzoyl peroxide (Martin and Ernst 2003). Tea tree oil is equally effective in treating acne compared with standard conventional therapy but does not produce the same side effects, such as irritation (Basseft, Pannowitz et al. 1990). In addition to its anti-acne effects, Tea tree oil has been shown to have anti-inflammatory properties (Koh, Pearce et al. 2002). There are many beneficial properties of tea tree oil (Mantle, Gok et al. 2001) specifically. The active components of Tea tree oil are effective in eradicating several types of bacteria, including the bacteria that cause acne, P. acnes (Raman, Weir et al. 1995). The forumulation presented in this invention includes a 4% tea tree oil concentration, however it should be noted that up to 50% tea tree oil could be formulated as well. Studies have shown that topical application of tea tree oil results in significant quantities of oil within the follicle where acne occurs (Biju, Ahuja et al. 2005).

In the present invention, Tea tree oil is used in combination with benzoyl peroxide or with or without adjunct traditional acne-fighting ingredients such as ascorbic acid and derivatives (eg., alpha.-hydroxy acids), benzoyl peroxide, salicylic acid, glycolic acid or retinoids. In these embodiments, tea tree oil is applied and then a conventional acne medication is applied. After daily treatment of the benzoyl peroxide and tea tree oil system (with or without additional compounds), improvement in the skin condition occurs. While conventional therapies can pose significant irritation problems and side effects, the application of natural anti-acne products induces minimal side effects and less irritation. In short, maximal efficacy is achieved with less irritation.

It is unique to this invention that, unlike all other manners of anti-acne treatment, tea tree oil is used in combination with benzoyl peroxide such that topical benzoyl peroxide concentration can be maximized. Skin tolerance is not known to previously exist with simply tea tree oil. Indeed, the anti-inflammatory properties used to counteract the benzoyl peroxide therapy are not likely to lose efficacy. In other words, effective anti-acne therapy is expected with high dose benzoyl peroxide and the anti-inflammatory property is expected to continue and not lose tolerance. Ideally, it is desirable to apply tea tree oil very often and in very high concentrations toward the beginning of treatment of acne-affected skin, simply to eradicate the acne as quickly as possible so that the patient does not have visible acne.

In the present invention, Tea tree oil is used in combination with the traditional acne-fighting ingredient benzoyl peroxide. This specific combination is optimal because:

1) Benzoyl peroxide remains the workhorse of topical over-the-counter therapies because of its longstanding effective cleansing and antibacterial properties.

2) High concentrations of benzoyl peroxide can be tolerated (or perceived to be tolerated) when combined with tea tree oil because tea tree oil has anti-inflammatory properties.

3) Anti-inflammatory properties of tea tree oil decrease the irritating side effects of benzoyl peroxide.

4) It is an added advantage that tea tree oil has anti-bacterial properties and therefore its effect is synergistic with benzoyl peroxide in fighting acne.

5) Because of the real or perceived benefit of these products in combination, there is increased compliance. The patient will follow instructions and apply the regimen more faithfully and not abandon the product, because they are seeing immediate results.

In the present invention, benzoyl peroxide is applied in combination with tea tree oil. After daily treatment with the tea tree oil in combination with the benzoyl peroxide, improvement in the skin condition occurs. While conventional therapies can pose significant irritation problems and side effects, the application of natural anti-acne products induces minimal side effects and less irritation. In short, enhanced perceived or actual efficacy is achieved with perceived or reduced irritation.

The amounts of benzoyl peroxide introduced to the skin can be increased dramatically, because the tea tree oil counteracts the irritation properties of the benzoyl peroxide. Thus, not only is the acne combated better with higher than normal levels of benzoyl peroxide, but the tea tree oil itself combats the acne. The result is an improved acne treatment product beyond that which is currently available today.

### REFERENCES

(2003). “Acne, isotretinoin and depression.” Drug Ther Bull 41(10): 76-8.
Barak, Y., Y. Wohl, et al. (2005). “Affective psychosis following Accutane (isotretinoin) treatment.” Int Clin Psychopharmacol 20(1): 39-41.
Bassett, I. B., D. L. Pannowitz, et al. (1990). “A comparative study of tea-tree oil versus benzoylperoxide in the treatment of acne.” Med J Aust 153(8): 455-8.
Biju, S. S., A. Ahuja, et al. (2005). “Tea tree oil concentration in follicular casts after topical delivery: determination by high-performance thin layer chromatography using a perfused bovine udder model.” J Pharm Sci 94(2): 240-5.
Biju, S. S., A. Ahuja, et al. (2005). “Formulation and evaluation of an effective pH balanced topical antimicrobial product containing tea tree oil.” Pharmazie 60(3): 208-11.
Bojar, R. A. and K. T. Holland (2004). “Acne and Propionibacterium acnes.” Clin Dermatol 22(5): 375-9.
Bremner, J. D., N. Fani, et al. (2005). “Functional brain imaging alterations in acne patients treated with isotretinoin.” Am J Psychiatry 162(5): 983-91.
Brinker, A., C. Kornegay, et al. (2005). “Trends in adherence to a revised risk management program designed to decrease or eliminate isotretinoin-exposed pregnancies: evaluation of the accutane SMART program.” Arch Dermatol 141(5): 563-9.
Honein, M. A., C. A. Moore, et al. (2004). “Can we ensure the safe use of known human teratogens? Introduction of generic isotretinoin in the US as an example.” Drug Saf 27(14): 1069-80.
Koh, K. J., A. L. Pearce, et al. (2002). “Tea tree oil reduces histamine-induced skin inflammation.” Br J Dermatol 147(6): 1212-7.
Mantle, D., M. A. Gok, et al. (2001). “Adverse and beneficial effects of plant extracts on skin and skin disorders.” Adverse Drug React Toxicol Rev 20(2): 89-103.
Martin, K. W. and E. Ernst (2003). “Herbal medicines for treatment of bacterial infections: a review of controlled clinical trials.” J Antimicrob Chemother 51(2): 241-6.
Morrison, D. G., F. J. Elsas, et al. (2005). “Congenital oculomotor nerve synkinesis associated with fetal retinoid syndrome.” J Aapos 9 (2): 166-8.
Raman, A., U. Weir, et al. (1995). “Antimicrobial effects of tea-tree oil and its major components on Staphylococcus aureus, Staph. epidermidis and Propionibacterium acnes.” Lett Appl Microbiol 21(4): 242-5.
Samuel, M., R. C. Brooke, et al. (2005). “Interventions for photodamaged skin.” Cochrane Database Syst Rev(1): CD001782.
Stamp, D. (2004). “Antibiotic therapy may induce cancers in the colon and breasts through a mechanism involving bile acids and colonic bacteria.” Med Hypotheses 63(3): 555-6.
Taylor, G. A. and A. R. Shalita (2004). “Benzoyl peroxide-based combination therapies for acne vulgaris: a comparative review.” Am J Clin Dermatol 5(4): 261-5.
Velicer, C. M., S. R. Heckbert, et al. (2004). “Antibiotic use in relation to the risk of breast cancer.” Jama 291(7): 827-35.
Wolverton S E: Comprehensive Dermatologic Drug Therapy Philadelphia, 2001, W.B. Saunders Company.

### DETAILED DESCRIPTION OF THE PRESENT INVENTION

Within this specification and claims therein, the term “acne” is intended to include all types of acne involving skin. It is noted that there are many clinical manifestations and variations of acne. It is the intention of this invention to address the clinical problem involving the skin including the hair follicles and oil glands in all stages of clinical presentation and in all ages. It is also noted that acne can go through multiple stages of development. In a mild clinical form, comedones (black and white heads) appear without redness and irritation. This early form is considered a “noninflammatory phase.” As the clinical acne condition becomes more inflamed and serious, pustules, and red bumps appear. The more inflammation present, the more likely the patient is prone to develop scars and discoloration changes in the skin. In even more severe forms, there is deep acne that leaves serious disfigurement. Anti-acne thereapy is usually directed toward either preventing, minimizing, eliminating, or decreasing the number of blackheads, whiteheads, pimples, pustules, inflammation, redness, red bumps, scars, discoloration, or disfigurement that is a result of this physiologic process therein.

It should be understood that there are various conventional ways of administering the formulation, and the present invention is not limited to one particular embodiment. The unique qualities of the present invention lie in the amount and combination of TTO and BPO in the formulation. There are various formulations described within this invention. In all formulations, the anti-inflammatory property of TTO is used to counteract the irritating effects of conventional therapies. It is an added advantage to this invention that TTO has anti-acne effects as well. In its basic formulation, the anti-inflammatory action of TTO is used in a 3-step system whereby BPO cleanser concentration can be maximized due to the anti-inflammatory component of TTO. This preliminary formulation is as follows:

### I. FACIAL CLEANSER, TONER, MOISTURIZER

The basic invention in its preferred embodiment involves using a 2- or 3-step system with a cleanser and lotion; a toner is added to the 3-step method. For each type of skin (dry, normal, oily), the system will be modified. The basic 3-step system is outlined as follows:

A) Normal Skin

a. Step 1: Glycol based cleanser consisting of benzoyl peroxide (up to 35%), propylene glycol and/or ethylene oxide polymers and nonionic surfactants such as the laureth and/or myreth amine oxides; fatty acid alkanolamides, alcohol ethoxylates, PEG-40 hydrogenated castor oil, nonoxynol 15, alkyl glucosides, PEG-7 glyceryl cocoate, propylene glycol caprylate, sorbitan oleate, and others. It is the intent of this invention that this cleanser is washed off with water. It should be noted that using benzoyl peroxide in 2.5% concentration is conventional in typical over the counter products. In this invention, higher concentrations are used).

b. Step 2: Toner containing tea trea oil up to 50% with or without witch hazel is applied by wash or spray or wiped on with a cotton pad or cotton ball after the face is washed and blotted dry. It is the goal of this step to shrink the pores and reduce the inflammation caused by the first step of the product. A preservative is added to prevent the growth of bacteria, yeast and mold. [Typical preservatives include, but are not limited to: phenoxyethanol, parahydroxybenzoate esters, sorbic acid, sodium benzoate, diazolidinyl urea, imidazolidinyl urea, iodo propynyl butyl carbamate, sodium hydroxymethyl glycinate, methyl dibromo glutaronitrile, benzalkonium salts]. Fragrance and coloring may be added as well as other ingredients for marketing purposes.

c. Step 3: Lotion is applied to increase moisturization of the skin that has a hydrator such as glycerol, propylene glycol, sodium PCA (pyrrolidine carboxylic acid), sodium hyaluronic acid and others that have a high water binding capacity. It may also have an occlusive agent that lowers trans epidermal water loss such as botanical butters (shea, cocoa, mango, avocado, etc.), squalane, botanical oils (jojoba, sunflower, avocado, sweet almond, avocado, kukui nut, macadamia nut, olive, etc.), beeswax, mineral oil and petrolatum are examples. Occlusive agents form a hydrophobic film on the skin's surface and prevent water from evaporating. Ingredients that stabilize the emulsion, thickeners, preservatives and fragrances are added. New ingredients may also be included in the moisturizer that are intended to provide anti-aging, anti-acne, or other special dermatological properties.

The formulations for Dry and Oily skin are mild modifications of the above product and final formulation may vary from that described here.

B) Dry Skin: for dry skin types, step 1 has a higher oil content compared with normal type skin, for increased soothing. Additionally, step 3 has a thicker moisturizing element so that dry skin can be treated with effective anti-acne therapy is provided.

a. Oil based cleansers containing 2.5% benzoyl peroxide are meant to be wiped off with a tissue or soft cloth, not washed off with water. They are composed mostly of mineral oil with a small amount of a mild nonionic and/or cationic surfactant. Botanical oils such as those from coconut, almond, jojoba, meadowfoam seed, kukui nut, macadamia nut, illipe nut, sunflower, rosehips, avocado, olive, grape seed, canola, soybean, safflower, etc. may be used in place of mineral oil.

b. Toner containing witch hazel.

c. Lotion and/or cream for increased moisturization.

C) Oily Skin:

For oily skin, the water and alcohol content is increased compared with normal skin. This serves to diminish the oil from the skin and minimize occlusion of pores.

a. Water-based cleanser, benzoyl peroxide (2.5%), 5-15% of one or more surfactants at least one of which is water-soluble and 5-25% of other ingredients (preservatives, moisturizers, fragrance, thickener or viscosity modifier, botanical extracts, and other ingredients to improve the dermatological properties and marketability of the formulation). These cleansers are to be washed off with water instead of being left on the skin. Numerous anionic, nonionic and cationic surfactants are commercially available that are used for this type of cleanser. Examples are: sodium, potassium, TEA or ammonium lauryl sulfate, sodium, potassium, TEA or ammonium laureth or myreth sulfate, sodium lauroyl glutamate, alkyl polyglycosides, alkyl glucosides, glyceryl cocoate, alkyl polygluco sulfosuccinates, alkyl polygluco citrates, alkyl polygluco tartrates, and phosphorylated nonionic surfactants.

b. Toner that contains witch hazel or an alcohol component.

c. Lotion for moisturization as above.

For the face: Patients using the product will be instructed to first gently remove any makeup from the skin and face. Then the patient should wash their skin with warm water and apply some of the cleanser to the palm of their hand. The cleanser should be washed over the face using the rubbing action of their hands or a washcloth, being careful not to get the product in their eyes, as the product will cause irritation. After approximately 15 seconds of cleansing, the person should rinse with warm water, pat the face dry, and apply a small amount of lotion (enough to cover the face). This step-wise manner is done in this way because inherent to acne treatment is unclogging the pores. First, an acne product must unclog pores with a wash. BPO is used in a wash because, if left in place, it would be irritating and would bleach the person's clothes or bed linens. After the wash is done and BPO is removed, the toner applies TTO, which soothes. In the present invention, the soothing helps diminish some of the irritation from the BPO, allowing the higher concentration of BPO in step 1. In the third step, some light moisturization takes place.

### II. BODYWASH

In an alternative embodiment of the present invention, the combination can be packaged as a bodywash to be used in the shower. After wetting the body with water, the product is lathered onto the chest, back, or other area in order to cleanse. Rinse liberally. Apply a light moisturizer. The product will be sold as a kit—a set of 2 or 3 for various uses on the body (eg, face, body, and beard formulations).

### III. ALTERNATIVE USES

In alternative embodiments of the present invention, the benzoyl peroxide (BPO) combination can be used or altered per the following:
as a shaving cream for people with follicular based beard acne
changing the combination to a bar formulation
for patients with comedonal-based acne, the product can be combined with topical retinoids
for patients with acne/rosacea combination, the product is further enhanced with metronidazole
for patients with pustular variants of acne, combination of the product is made with topical clindamycin antibiotic
combined with anti-aging properties, such as glycolic acids
more severe acne, vitamin c derivatives can be added.

In additional embodiments of the present invention, the product could be produced in various forms, such as a spray, impregnated pad, sponge, body bath, exfoliating scrub, or even combined with oatmeal or other all-natural application. It is conceivable that the synergistic effect of using BPO and TTO together are further enhanced by other known anti-acne therapies. Combinations may even further enhance efficacy.

### IV. MAIN INGREDIENTS OF THE PRESENT INVENTION

The composition may be a clear, opaque, aqueous, or alcohol based solution, which may comprise surfactant, humectant, emulsifier or solubilizing agent. Additionally the oil(s) may be applied in a pad or impregnated solution singly or together. These formulations may be applied as a wash, lotion, spray, toner, or exfoliant. The constituents involved in fighting the acne include the terpinen constituents, that include alpha-terpineol, terpinen-4-ol, alpha-pinene.

Compositions of the invention may be formulated alone in this system or combined with traditional acne-fighting ingredients such as ascorbic acid and derivatives, benzoyl peroxide, salicylic acid, glycolic acid and other alpha-hydroxy acids, or retinoids. Adjunct ingredients include but are not limited to alpha-hydroxy acids, fatty acid esters of ascorbic acid, vitamin A and vitamin A derivatives.

Formulations of the invention may contain DMAE for additional anti-inflammatory activity. Additionally, topical DMAE is used in currently marketed anti-acne regimens.

### V. SECONDARY INGREDIENTS OF THE PRESENT INVENTION

The following are possible combinations to be added to the main ingredients of the present invention.

1. Azelaic acid: The present invention may be combined with azelaic acid, a naturally occurring anti-acne treatment. Azelaic acid is effective at reducing the bacterial load on the skin and also decreases the color changes that can occur. There are several products that are currently marketed in the United States that contain azelaic acid. The side effect of azelaic acid is dryness and it can lighten non-affected areas of the skin. The azelaic acid must therefore be used in combination with other compounds to minimize the effect of dryness or lightening of the skin.

2. Alpha-hydroxy acid (AHA): The term “alpha-hydroxy acid” (AHA) describes a class of an organic compound that contains a hydroxyl group on the carbon atom bonded to or located immediately next to the carboxyl group carbon. There are several types of alpha-hydroxy acids. Commonly used ones include: glycolic acid, lactic acid and citric acid as well as others. The smallest chemical structure alpha-hydroxy acid is glycolic acid. Used in aqueous solution, glycolic acid is stable and often therefore used in combination with other anti-acne products. There are several actions of AHA. The anti-acne property of AHA appears to be partly from the exfoliating features. The skin has several layers and the most superficial layer, the stratum corneum, is partially removed which helps at unclogging pores and diminishing acne. AHA's also have a rejuvenating effect and can change the biochemical features of the skin, increasing the youthful appearance. As well, photodamage is reversed; AHA's have a therapeutic effect against premalignant sun damage spots (called “actinic keratosis”). It should be noted that the invention encompasses formulations with AHA.

It should be noted that Glycolic acid is safe; if ingested it is nontoxic. In 1997, the FDA Office of Cosmetics FDA concluded that glycolic acids are safe in over-the-counter retail industry in preparations equal to or less than 10% if the pH of the product is greater than or equal to 3.5. Physician-supervised formulations include preparations that exceed the 10%. Formulations of 35 to 70% are not uncommon. The usual percent alpha-hydroxy acid ranges from 1 to 10% glycolic (or other AHA). The present invention may also comprise formulations that combine AHA's, or include AHA in subsequent steps. It is noted that the anti-inflammatory properties of the invention may counter AHA's most serious side effect—irritant dermatitis. AHA's are included in many over-the-counter products currently marketed as MD Forte, Glytone, Neostrata and Glyderm.

4. Vitamin A: Vitamin A derivatives such as retinol have been shown to be effective in anti-acne as well as anti-aging treatments. There are several over-the-counter therapies that combine retinol with moisturizers, creams, anti-acne treatments, night creams, as well as the above-described AHA. The present invention includes formulations that incorporate a retinol as a step or product. Examples of currently marketed products include: Retinol Energizing Moisturizer (DDF), Retinol Complex (SkinMedica), Retinol Refining Night Cream (SkinCeuticals), Replenix Retinol Smoothing Serum (Topix), Max Retinol Wrinkle Repair (Peter Thomas Roth). As well, currently available and marketed retinol products include other rejuvenating and anti-acne therapies. For instance, A+ Retinol Serum (Institut DERMed) incorporates a glycolic with the retinol. Other Vitamin A derivatives include retinyl palmitate, retinyl palitate, retinoic acid, retinal, and retinyl propionate.

5. Retinoids: Retinoids comprise a class of compounds that provide exfoliating, or desquamative, properties. Shown to be extremely effective in treating acne, the side effect of irritation limits their use by many people. For some, discontinuance of the product occurs. Types of topical retinoids that are available by prescription include adapalene, tazarotene and/or tretinoin. Various names of topical retinoid include, but are not limited to, the following: Altinac (marketed by Upsher-Smith Laboratories), Avita (Bertek Pharmaceuticals), Renova (Ortho McNeil Pharmaceutical), and Retin-A (Ortho Dermatological).

6. Vitamin C: Vitamin C and its derivatives have been shown to have anti-acne action and to stabilize active anti-acne ingredients. There are many derivatives of Vitamin C; they are fatty ester derivatives. Several types include but are not limited to the ascorbyl behenate, ascorbyl laurate, ascorbyl myristate, ascorbyl palmitate, ascorbyl stearate, and other derivatives of Vitamin C. The invention incorporates formulations with Vitamin C or its derivatives. There are many ways to prepare the esters. In one type of processing, a hydrogenated fat or oil is used. Additionally, other types of esters can be combined with the Vitamin C or derivative. It should be noted that Vitamin C has anti-acne as well as preservative functions. For the present formulation, Vitamin C can be used to stabilize ingredients as well as be used as an adjunct in treating acne.

7. Other combinations: As stated previously, the invention can be combined with other anti-acne or other cosmetic products, such as makeup, sunscreen, tinting agents, anti-aging treatments.

8. Antibiotics: In the present invention, topical or oral antibiotics, especially in patients who have more extensive or severe acne can be combined with the invention's treatment. Examples of antibiotics that are commonly used for acne include, but are not limited to, the tetracycline family, which includes tetracycline, doxycycline, minocycline. Some commonly used brand names in the United States are:
Tetracycline
Sumycin
Terramycin™
Doxycycline
Doryx™ Warner Chilcott, USAWarner Chilcott Laboratories
Vibra-Tabs™ Pfizer Inc
Vibramycin™
Minocycline
Dynacin™ Medicis
Minocin™
Declomycin™
Monodox™

Additionally, clindamycin and erythromycin are often used. Examples of Topical Clindamycin:
Cleocin T™ is a Gel, lotion, and topical solution.
Clinda-Derm

Many formulations of topically applied antibiotic include benzoyl peroxide. Examples of topical clindamycin antibiotic marketed in combination with benzoyl peroxide include Benzaclin and Clindagel and Duac.

In the above discussion, the side effects of antibiotics were discussed; particularly their adverse effects. Long term use of antibiotics has the risk of possible susceptibility of breast cancer. Additionally, long term antibiotics can increase bacterial resistance. Similar serious side effects can occur with topical antibiotics as well.

9. Sodium sufacetamide: Sodium sufacetamide is a treatment for acne that uses a sulfur-based compound that has efficacy against acne. It is a key ingredient in many over-the-counter and prescription treatments. Although it is effective, a malodorous experience causes many people to discontinue its use. As well, it is not suitable for people who have allergies to sulfa medications.

10. Possible Diluents: soap, cleanser, wash, cream, lotion, gel, emulsion, micro-emulsion, spray, impregnation in soaked pad. As mentioned before, there are several delivery vehicles for the present invention. Emollients, surfactants, buffers, anti-oxidants, pads, impregnating substances, emulsifiers, alcohols, water, can all be used with the formulation of the present invention.

11. Emollients: The present invention encompasses formulations whereby the active ingredient(s) are dissolved or saturated into emollients. Emollients are usually used in cosmetic products to soften and protect the surface of the skin. There is usually an oily component to the emollient. The emollient is mixed with water to form an emulsion so that the oil is effectively delivered and coated onto the skin. There are several types of emollients used in common cosmetic practice. Fatty esters, fatty alcohols, mineral oils, and polyether siloxane copolymers are the usual varieties. There are synthetic as well as natural emollients. The present invention encompasses emollients that include either synthetic, natural, or both types of emollient. Examples of synthetic emollients include: Polyethylene Glycol compounds (eg PEG-45 Almond Glyceride as well as others), long chain fatty alcohols (for instance chemicals containing the phrase cetearyl-, cetyl-, myristyl-, octyl-, dodecyl- or stearyl, as well as others). Other types of emollients include paraffin, petrolatum, and silicone as well as mineral oil, dimethicone, cyclomethicone and copolyol. Natural emollients include many plant oils including but not limited to Jojoba, avocado and rosehip. Vitamin E, also called alpha-tocopherol and its esters such as tocopheryl acetate and tocopheryl palmitate are also emollients. Lanolin is an emollient extracted from wool. Botanical butters such as Cocoa, Jojoba, shea, illipe, olive, mango and avocado are also good emollients.

12. Humectants: Humectants are used in commonly marketed products to keep the skin moist. There are many types of humectants that can be used in the present invention. Common types include polyhydric alcohols (butylenes glycol, glycerol (also called glycerin), diethylene glycol, dipropylene glycol, glycerol, polypropylene glycol (PPG), polyethylene glycol (PEG), propylene glycol), panthenol (pro-Vitamin B5), sodium PCA (pyrrolidone carboxylic acid), hyaluronic acid and its salts, alpha hydroxyl acids (AHA), hydrolyzed proteins (amino acids and peptides), sodium polyaspartate, locust bean gum, lecithin, and its derivatives.

13. Emulsifier: There are many types of emulsifiers that can be used in the present invention. Alkoxylated Amides, PEG compounds, Sorbitan and its derivatives, including but not limited to: Stearate, Laurate, Palmitate, Oleate; stearic acid, waxes, silicone, stearates, Stearyl-, Cetyl-, Ceteareth or Steareth-alcohols as well as lanolin, lecithin, all are used to emulsify commonly used cosmetics. Natural cosmetic products use plant waxes as emulsifiers combined with nature-derived thickeners like xanthan gum and carrageenan.

14. Preservatives: The present invention includes formulations with natural as well as artificial preservatives. Examples of preservatives include, but are not limited to: Benzalkonium Chloride, Butylated Hydroxytoluene (BHT), Butylated Hydroxyanisole (BHA), Butylparaben, DMDM Hydantoin, Chloromethylisothiazolinone, Diazolidinyl urea, Ethylparaben, Imidazolidinyl urea, Methylisothiazolinone, Methylparaben, Phenoxyethanol, Propylparaben, Quaternium 15, 2-Bromo-2-Nitro-Propane-1,3-diol. There are natural products as well, which include Thyme essential oil, Rosemary Extract, Grapefruit seed extract and Vitamin E (Tocopherol).

15. Surfactants:

A) Some Synthetic Surfactants
Sodium or Ammonium Lauryl or Laureth Sulphate
Sodium Methyl Cocoyl Taurate
Sodium Lauroyl or Cocoyl Sarcosinate
Cocomidopropyl Betaine
TEA compounds (TriEthanolAmine), DEA—DiEthanolAmine and MEA MonoEthanolAmine
DEA compounds (DiEthanolAmine)
MEA compounds (MonoEthanolAmine)
PEG (Polyethylene Glycol) compounds
Quarternium-7,15,31,60 etc
Lauryl or Cocoyl Sarcosine
Disodium Oleamide or Dioctyl Sulfosuccinate

B) Some Natural Surfactants
Castile Soap
Yucca Extract
Soapwort
Quillaja Bark Extract

16. Perfumes

17. Antioxidants
BHT
VitaminC (ascorbic acid)
Ascorbyl palmitate and other vitamin C derivatives
BHA
Phenyl-alpha naphthylamine
Hydroquinone
Propyl gallate
Nordihydro-quiaretic acid
Vitamin E
Vitamin E derivatives
Tocotrienol
Alpha Lipoic Acid
Green Tea
Beta Carotene
Lutein
Astaxanthin
Zeaxanthin
Selenium
Lycopene

These examples are not meant to limit the formulation of the present invention to the embodiments as listed above.

### VI. EXAMPLES

The following examples are presented to further illustrate and explain the present invention and should not be taken as limiting in any regard. Unless otherwise indicated, all percentages are by weight of the total composition, except tea tree oil, which are by volume.

### Example 1

A preferred formulation is a wash, toner and moisturizer. Since high concentrations of BPO are used, the TTO and moisturizer provides a soothing function to counteract the irritating effects of TTO.

Other embodiments are possible as described in previous sections and within the scope of the following claims. Any conventional delivery system is possible.

What is claimed is:

1. A product for the treatment of acne on a user's skin, comprising:
tea tree oil; and
benzoyl peroxide.

2. The product of claim 1 wherein said tea tree oil is greater than 0.05% by volume.

3. The product of claim 1 wherein said tea tree oil is less than 4% by volume.

4. The product of claim 1 wherein said tea tree oil is greater than 0.05% by volume and less than 4% by volume.

5. The product of claim 1 wherein said benzoyl peroxide is greater than 2.5% by volume.

6. The product of claim 1 wherein said benzoyl peroxide is less than 35% by volume.

7. The product of claim 1 wherein said benzoyl peroxide is greater than 2.5% by volume and less than 35% by volume.

8. The product of claim 2 wherein said benzoyl peroxide is greater than 2.5% by volume.

9. The product of claim 2 wherein said benzoyl peroxide is less than 35% by volume.

10. The product of claim 2 wherein said benzoyl peroxide is greater than 2.5% by volume and less than 35% by volume.

11. The product of claim 3 wherein said benzoyl peroxide is greater than 2.5% by volume.

12. The product of claim 3 wherein said benzoyl peroxide is less than 35% by volume.

13. The product of claim 3 wherein said benzoyl peroxide is greater than 2.5% by volume and less than 35% by volume.

14. The product of claim 4 wherein said benzoyl peroxide is greater than 2.5% by volume.

15. The product of claim 4 wherein said benzoyl peroxide is less than 35% by volume.

16. The product of claim 4 wherein said benzoyl peroxide is greater than 2.5% by volume and less than 35% by volume.

17. The product of claim 1 further comprising conventional acne medication including salicyclic acid or tretinoin.

18. The product of claim 1 wherein said tea tree oil and said benzoyl peroxide are applied to the user's skin independently of one another.

19. The product of claim 1 further comprising a hydrator.

20. The product of claim 1 further comprising an occlusive agent.
